(19) United States
(12) Patent Application Publication
(10) Pub. No.: US 2016/0360997 A1
(43) Pub. Date: Dec. 15, 2016

(54) SYSTEMS AND METHODS FOR MEASURING RELATIVE ORIENTATION AND POSITION OF ADJACENT BONES

(71) Applicant: MIRUS LLC, Atlanta, GA (US)

(72) Inventors: Jay Yadav, Atlanta, GA (US); Angad Singh, Atlanta, GA (US)

(73) Assignee: MIRUS LLC, Atlanta, GA (US)

(21) Appl. No.: 15/120,814

(22) PCT Filed: Feb. 23, 2015
(86) PCT No.: PCT/US15/17158

§ 371 Date: Aug. 23, 2016

Related U.S. Application Data
(60) Provisional application No. 61/943,493, filed on Feb. 23, 2014.

(51) Int. Cl. A61B 5/107; A61B 5/00; A61B 5/11

(52) U.S. Cl. CPC A61B 5/1072; A61B 5/1121; A61B 5/4528; A61B 5/4571; A61B 2562/0223; A61B 2562/0219

(57) ABSTRACT

A method for estimating leg length and offset, comprises registering an anatomic coordinate frame associated with a patient's pelvis. The method also comprises measuring a first position of a femur relative to the patient's pelvis. The method further comprises receiving, from magnetic and orientation sensors, information indicative of a change in a position of the femur relative to the pelvis of the patient's pelvis. Alternatively, the method further comprises receiving, from light and orientation sensors, information indicative of a change in a position of the femur relative to the pelvis of the patient's pelvis. The method also comprises determining at least one of a leg length and an offset based on the first position and the change in position.

### TECHNICAL FIELD

The present disclosure relates generally to orthopedic surgery and, more particularly, to an apparatus and method for intra-operatively measuring prosthetic placement parameters during orthopedic arthroplastic procedures.

### BACKGROUND

Orthopedic procedures involving resurfacing, replacement, or reconstruction of joints using multi component prosthesis with articulating surfaces. In such procedures proper placement of the prosthetic component is critical for longevity of the implant, positive clinical outcomes, and patient satisfaction.

Currently, many orthopedic surgeons intra-operatively evaluate prosthetic component placement using an imprecise combination of subjective experience of the surgeon and rudimentary mechanical instrumentation. For example, in hip replacement surgery, there are three parameters that are typically used to quantify differences in prosthetic joint placement: leg length (also called hip length), offset, and anterior/posterior position. Leg length refers to the longitudinal extent of the leg measured in the superior/inferior axis relative to the pelvis. Offset refers to the position of the leg in the medial-lateral axis relative to the pelvis. Anterior/posterior (“AP”) position of the leg, as the name suggests, refers to position of the leg along the anterior/posterior axis with respect to the pelvis.

Early methods for calculating leg length, offset, and anterior/posterior position required the surgeon to use rulers and gauges to perform manual measurements on the hip joint before and after attaching the prosthetic implants. Such measurements, however, are often inaccurate due to the difficulty in performing manual measurements in the surgical environment using conventional rulers and gauges. Further, manual measurements are not easily repeatable or verifiable, and can take a significant amount of time to perform.

Because existing techniques for intra-operative evaluation are extremely subjective and imprecise, the performance of the reconstructed joint is highly variable and dependent on the experience level of the surgeon. Perhaps not surprisingly, it is difficult for patients and doctors to reliably predict the relative success of the surgery (and the need for subsequent corrective/adjustment surgeries) until well after the initial procedure. Such uncertainty has a negative impact on long term clinical outcomes, patient quality of life, and the ability to predict and control costs associated with surgery, recovery, and rehabilitation.

Some computer/robotically-assisted surgical systems provide a platform for more reliably estimating prosthetic placement parameters. These systems typically require complex and sophisticated tracking equipment, bulky markers/sensors, time-consuming instrument calibration/registration procedures, and highly-specialized software packages that often require technical support personnel to work with doctor in the operating room. Not only do such systems tend to be costly, they also tend to be far too complex to warrant broad adoption among orthopedic surgeons.

To overcome the accuracy and reliability issues associated with manual methods for determining joint placement parameters, while providing a cost-effective and relatively user-friendly approach that is unavailable in computer/robotically-assisted systems, a cost-effective, portable, and user-friendly tool and associated methods for measuring prosthetic component positioning would be advantageous. The presently disclosed prosthetic component positioning tool and associated methods for intra-operatively measuring joint placement parameters during orthopedic arthroplastic procedures are directed to overcoming one or more of the problems set forth above and/or other problems in the art.

### SUMMARY

According to one aspect, the present disclosure is directed to a method for estimating leg length and offset, and comprises registering an anatomic coordinate frame associated with a patient's pelvis. The method also comprises measuring a first position of a femur relative to the patient's pelvis. The method further comprises receiving information indicative of a change in a position of the femur relative to the pelvis of the patient's pelvis. The method also comprises determining change in at least one of a leg length and an offset based on the first position and the change in position.

In accordance with another aspect, the present disclosure is directed to a system for estimating leg length and offset associated with a patient's joint. The system comprises a magnetic field generator coupled to a patient's pelvis and configured to generate a magnetic field. The system also comprises a plurality of magnetic sensors coupled to a patient's femur and configured to measure the relative strength and direction of the magnetic field at the second location from which the relative position and/or orientation of the patient's femur is then calculated. The locations of the magnetic sensors and magnetic field generator may be interchanged without impacting the invention in any way. The system further comprises orientation sensors coupled to both locations and configured to detect information indicative of an orientation of the location relative to the patient's anatomy as well relative orientation between the two locations. The system also comprises a processor, communicatively coupled to the orientation sensors, magnetic sensors, and a magnetic field generator. The processor may be configured to register an anatomic coordinate frame associated with a patient's pelvis. The processor may also be configured to measure a first position of a femur relative the patient's pelvis. The processor may also be configured to receive information indicative of a change in a position of the femur relative to the patient's pelvis, and determine at least one of a leg length and an offset based on the first position and the change in position. In an alternate embodiment of the present disclosure, in addition to or in lieu of magnetic measurement of position, optical measurement of position is utilized to further improve the performance of the system. In such a system a light source, such as a laser or light emitting diode, and light detectors are utilized as part of an optical distance measurement system. The optical distance measurement is combined with the orientation measurements by the orientation sensors and/or the magnetic measurements from the magnetic sensors to calculate the relative 3D positions of the adjacent bones with a high degree of accuracy.

### BRIEF DESCRIPTION OF THE DRAWINGS

FIG. 1 provides a front view of a portion of an exemplary hip joint, the type of which may be involved in a joint replacement procedure consistent with certain disclosed embodiments;

FIG. 2A provides a schematic view of exemplary components associated with a prosthetic hip joint, which may be used in a joint replacement procedure consistent with the disclosed embodiments;

FIG. 2B illustrates a magnified view of an exemplary prosthetic hip joint in a reduced state in accordance with certain disclosed embodiments;

FIG. 3A provides a diagrammatic view of an exemplary sensor system used to measure leg length and offset during an orthopedic (hip) arthroplastic procedure consistent with certain disclosed embodiments;

FIG. 3B illustrates one exemplary principle of operation of the exemplary sensor system illustrated in FIG. 3A in accordance with certain disclosed embodiments;

FIG. 3C provides a diagram illustrating exemplary operations associated with the sensor system illustrated in FIG. 3A consistent with the disclosed embodiments;

FIG. 4 provides a schematic view of exemplary components associated with a leg length and offset monitoring system, such as that illustrated in FIG. 3A;

FIG. 5A illustrates an exemplary position of a registration tool during a registration process that involves estimating an orientation of a first virtual plane associated with a virtual coordinate position associated with a pelvis, consistent with certain disclosure embodiments;

FIG. 5B illustrates an exemplary position of registration tool during the registration process that involves estimating orientation of a second virtual plane associated with the virtual coordinate position associated with the pelvis, in accordance with certain disclosed embodiments;

FIG. 6 illustrates exemplary anatomical planes associated with the virtual coordinate system of the pelvis, the orientations of one or more of which may be estimated by processes consistent with the disclosed embodiments;

FIG. 7 provides a schematic view of exemplary components associated with an alternate embodiment of leg length and offset monitoring system, such as that illustrated in FIG. 3A;

### DETAILED DESCRIPTION

Systems and methods consistent with the embodiments disclosed herein are directed to a sensor-based system to measure changes in leg length and offset in a hip arthroplasty procedure. The system combines magnetic and inertial sensing to overcome inherent deficiencies of the individual sensing modalities leading to improved performance and robustness. In an alternate embodiment, the system combines optical, inertial sensing, and/or magnetic sensing to further improve performance. The system does not rely on expensive tracking or robotic equipment. Systems and methods consistent with the disclosed embodiments also limit the number of hardware components and steps needed to calibrate the system for use, potentially reducing the time and cost burden associated with the procedure. Certain exemplary embodiments can be described as “imageless,” meaning that they do not rely on any pre-operative or intra-operative imaging (X-ray, CT or MRI), which can add additional time and cost to the procedure and subject the patent to unnecessary exposure to potentially harmful radiation.

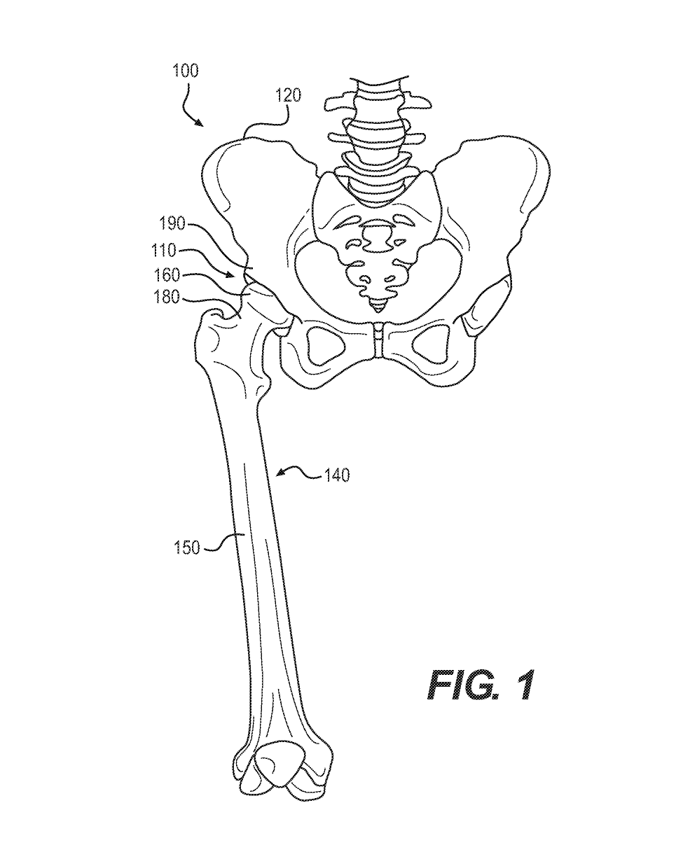

FIG. 1 illustrates a front view of an exemplary portion of the pelvic region 100 of the human body, which includes a hip joint 110. Proper articulation of hip joint 110 contributes to many basic structural and motor functions of the human body, such as standing and walking. As illustrated in FIG. 1, hip joint 110 comprises the interface between pelvis 120 and the proximal end of femur 140. The proximal end of femur 140 includes a femoral head 160 disposed on a femoral neck 180. Femoral neck 180 connects femoral head 160 to a femoral shaft 150. Femoral head 160 fits into a concave socket in pelvis 120 called the acetabulum 190. Acetabulum 190 and femoral head 160 are both covered by articular cartilage (not shown) that absorbs shock and promotes articulation of hip joint 110.

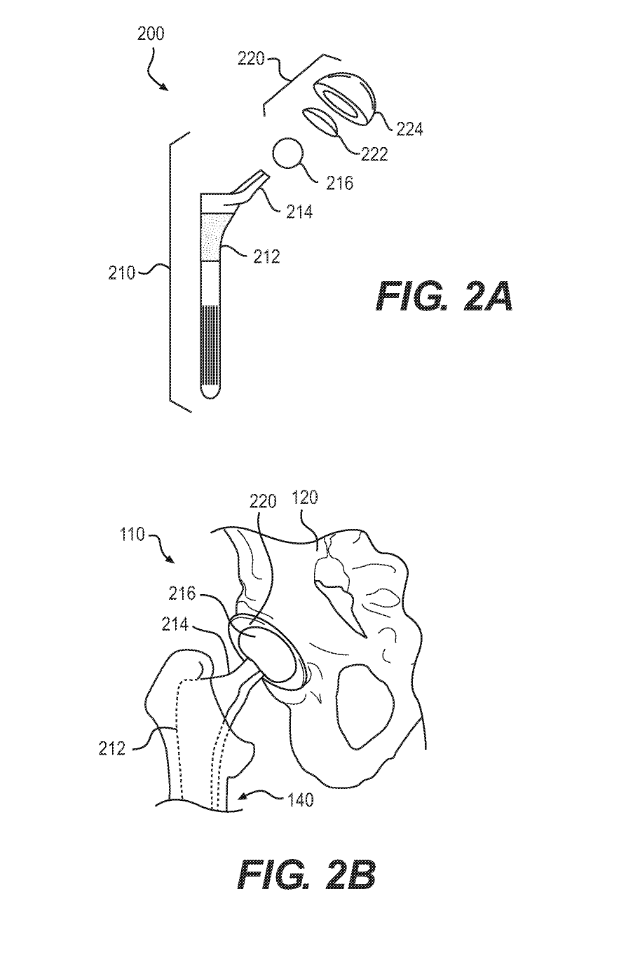

Over time, hip joint 110 may degenerate (due, for example, to osteoarthritis) resulting in pain and diminished functionality of the joint. As a result, a hip replacement procedure, such as total hip arthroplasty or hip resurfacing, may be necessary. During a hip replacement procedure, a surgeon may replace portions of hip joint 110 with artificial prosthetic components. For example, in one type of hip replacement procedure—called total hip arthroplasty (THA)—the surgeon may remove femoral head 160 and neck 180 from femur 140 and replace them with a femoral prosthesis. Similarly, the surgeon may resect or resurface portions of acetabulum 190 using a surgical reamer or reciprocating saw, and may replace the removed portions of acetabulum 190 with a prosthetic acetabular cup. Prosthetic components associated with the hip joint 110 are illustrated in FIG. 2A.

As illustrated in FIG. 2A, the natural (or “native”) femoral components removed during the arthroplasty may be replaced with a prosthetic femoral component 200 comprising a prosthetic head 216, a prosthetic neck 214, and a stem 212. Stem 212 of prosthetic femoral component 200 is typically anchored in a cavity that the surgeon creates in the intramedullary canal of femur 140.

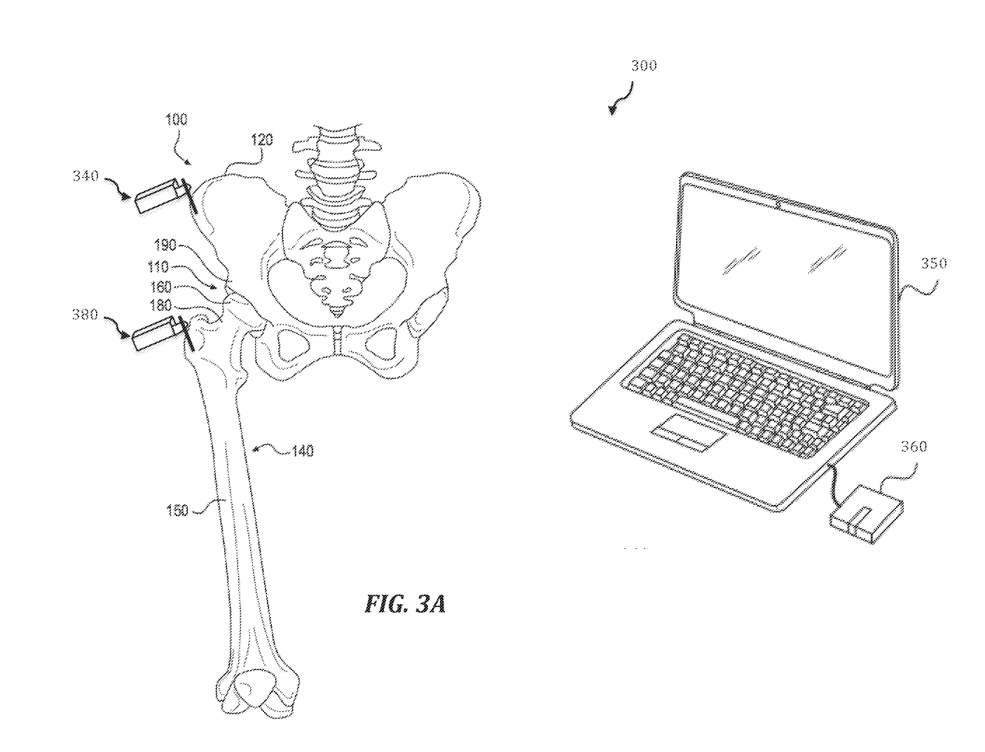

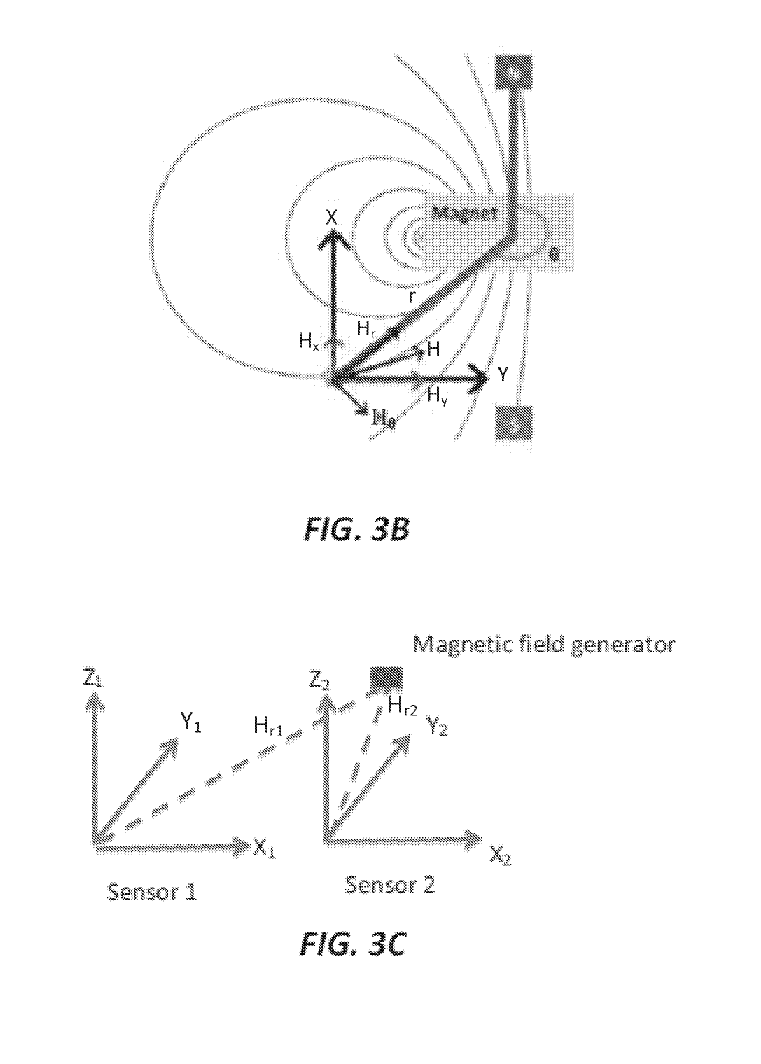

Similarly, the native acetabular components removed during the hip replacement procedure may be replaced with a prosthetic acetabular component 220 comprising a cup 224 that may include a liner 222. To install acetabular component 220, the surgeon connects cup 224 to a distal end (312 of FIG. 3) of an impactor tool (310 of FIG. 3) and implants cup 224 into the reamed acetabulum 190 by repeatedly applying force to a proximal end (313 of FIG. 3) of the impactor tool 310. If acetabular component 220 includes a liner 222, the surgeon snaps liner 222 into cup 224 after implanting cup 224 within acetabulum 220.

FIG. 2B illustrates a magnified view of an exemplary prosthetic hip joint in a reduced (i.e., assembled) state. As illustrated in FIG. 2B, the stem 212 is secured within the intramedullary canal of femur 140. The prosthetic head 216 is engaged with the acetabular component 220 of pelvis 120 to form the new prosthetic joint.

FIG. 3A provides a view depicting exemplary a hip surgical system to measure leg length and offset. However, the system can also measure A-P position and similar systems for other types of surgeries and measuring other parameters can be envisioned. As illustrated in FIG. 3A, the hip surgical system provides a solution for registering an anatomic coordinate frame such as of the pelvis, measuring the pre-dislocation position of the femur relative to the pelvis and storing this position as a reference, and measuring the changes in leg length (LL) and offset (OS) from the reference position during and after the joint reduction process and displaying this information in real-time.

As illustrated in FIG. 3A, the system 300 comprises a pelvic module 340 and femoral module 380 coupled to a processing and display unit 350. The pelvic and femoral modules may be interchanged without impacting the invention in any way. The femoral module 380 is rigidly attached to the femur and comprises at least two 3-axis magnetic sensors that measure the direction and intensity of a magnetic field. The pelvic module 340 is rigidly attached to the pelvic and comprises a magnetic field generator. In an example embodiment, the magnetic field generator may be a permanent magnet or electromagnet (i.e. a wound coil through which current is passed). In the case of an electromagnet, one or more coils may be utilized to create multiple magnetic fields that are at known orientations to each other. These fields can be created in sequence by passing currents at fixed intervals. For example 3 orthogonal coils may be utilized to create 3 orthogonal magnetic fields. A plurality of fields that are pulsed in sequence can allow the magnetic sensors to isolate the generated field from interfering fields like the earth's magnetic field thereby improving accuracy and precision

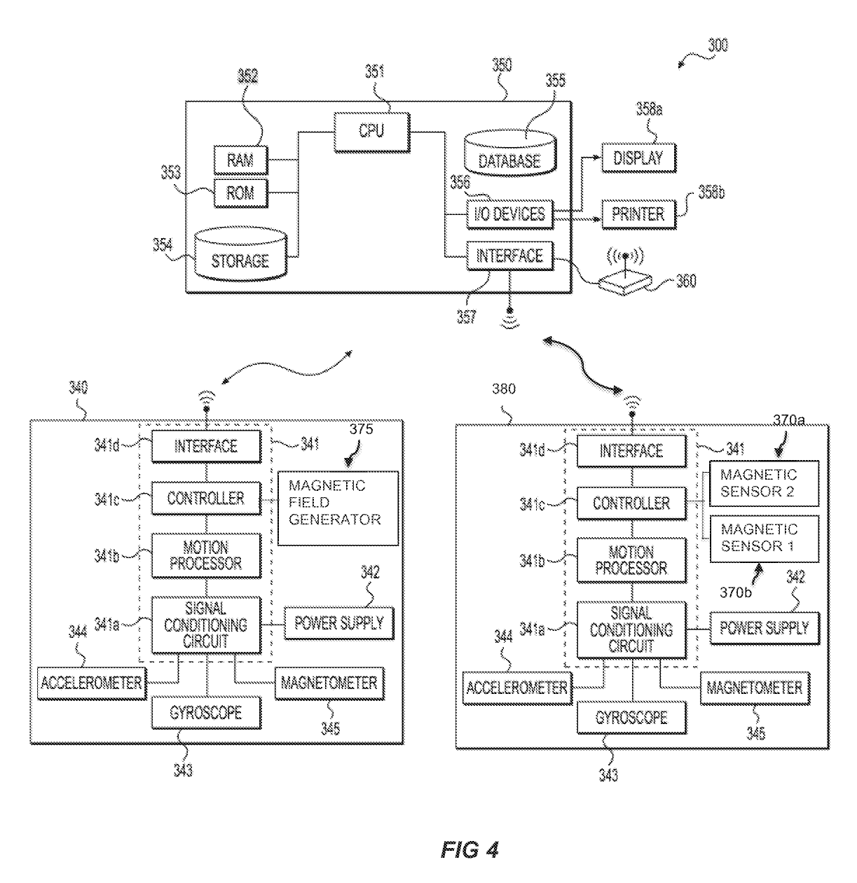

The pelvic module 340 and femoral module 380 may also include one or more inertial measurement units (IMUs). According to one embodiment, IMUs may include or embody one or more of 3-axis gyroscopes and 3-axis accelerometers, which are also fixed to each bone. The IMU may measure rotational motion and/or orientation in a reference coordinate frame or relative to a starting position or another IMU. The pelvic and femoral modules may be attached to the bone using pins or screws commonly used in orthopedic surgery. Inertial measurement units consistent with the disclosed embodiments are described in greater detail below with respect to the schematic diagram of FIG. 4.

The pelvic module 340 and femoral module 380 associated with presently disclose system may each be configured to communicate wirelessly with each other and to a processing and display unit 350 that can be a laptop computer, PDA, or any portable or desktop computing device. The wireless communication can be achieved via any standard radio frequency communication protocol such Bluetooth, Wi Fi, ZigBee, etc., or a custom protocol. In some embodiments, wireless communication is achieved via wireless communication transceiver 360, which may be operatively connected to processing and display unit 350.

The processing and display unit 350 runs software that calculates the LL and OS changes based on the sensor readings and displays the information on a screen in a variety of ways based on surgeon preferences. The surgeon or surgical assistants can interact with the processing unit either via a keyboard, wired or wireless buttons, touch screens, voice activated commands, or any other technologies that currently exist or may be developed in the future.

The magnetic sensors for measuring leg length and offset are configured to operate according to the magnetic field principles illustrated in FIG. 3B. As shown in FIG. 3B, the magnetic field emanating from a magnetic field generator such as a permanent magnet can be fully described using 2 degrees of freedom since the field is symmetrical about the magnet's pole axis. The field space can be described as a plane called the “magnetic field space.” Any point in the field space can be represented in polar form (r, θ), where r is the distance between the magnet and the sensor and θ is the angle between the sensor and magnets north pole. The magnetic field vector H can therefore be converted into its tangential and radial components Hᵣ and H_{θ}. These orthogonal vectors describe the position in the magnetic field space and need to be converted to the sensor's coordinate X,Y,Z frame as Hₓ and H_{y} as shown in FIG. 3B. This is only possible if the orientation of the magnetic field space in the sensor coordinate frame is known.

In the embodiments described herein, multiple 3-axis magnetic sensors are used to measure the direction and intensity of the magnetic field emanating from the magnetic field generator. These multiple magnetic sensors are arranged at a known fixed spatial distance and orientation with respect to each other. For example, as illustrated in FIG. 3C, they can be arranged with their X, Y, Z axis aligned and separated at a fixed distance along the X-axis.

In one embodiment, measured fields at these multiple magnetic sensors are utilized by a tracking algorithm to converge to a solution for the magnetic field generators X, Y, Z position that satisfies that requirement of the known spatial arrangement between the sensors. This information may be supplemented by the IMU's that are placed on both bones at known orientations from the magnetic sensors and magnetic field generator. The IMU's give complementary and overlapping information with regard to the relative orientation between the magnetic sensors and magnetic field generator. This information is combined with the information from the magnetic sensor to reduce uncertainty in the measurement and improve accuracy and precision. The information from the IMU can also assist with compensation for magnetic interference (hard and soft). Data from all the sensors as described above can be “fused” using any of the data filtering and data fusion methods known in the art such as a Kalman filter.

In another embodiment, the IMU's are used to measure the relative orientation between the magnetic sensors and magnetic field generator and this information is directly utilized to determine the orientation of the magnetic field space with respect to the sensors coordinate reference frame. As described earlier, this information is utilized in converting the magnetic field vector H into the sensor coordinate reference frame.

In addition to their role as described above, IMU's also allow a means for the system to register the anatomic planes (described later in this document). This registration allows conversion of the magnetic field generator's position in the sensor's X,Y,Z frame to a position in an anatomic reference frame.

In accordance with certain embodiments, the magnetic sensors may include or embody 3-axis Hall effect magnetic sensors or 3-axis magneto resistive sensors. Also, in certain exemplary embodiments, each IMU consists of 3-axis acceleration (accelerometers) and 3-axis angular rate (gyroscope) sensors and, in some cases, a 3-axis magnetic compass. The accelerometers, gyroscopes, and optional compass in the IMU work collectively to provide an accurate estimate of angular motion that can be processed to calculate orientation relative in a reference coordinate frame.

FIG. 4 provides a diagrammatic illustration of an exemplary system 300 for intra-operatively leg length, offset, and other joint performance parameters during orthopedic arthroplastic procedures, such as a replacement procedure for hip joint 110. FIG. 4 provides a schematic diagram illustrating certain exemplary subsystems associated with system 300 and its constituent components. Specifically, FIG. 4 is a schematic block diagram depicting exemplary subcomponents of processing and display unit 350, pelvic module 340, and femoral module 380 in accordance with certain disclosed embodiments. Those skilled in the art will recognize that embodiments consistent with the presently disclosed systems and methods may be employed in any environment involving arthroplastic procedures, such as the hip, knee and shoulder.

For example, in accordance with the exemplary embodiment illustrated in FIG. 4, system 300 may embody a system for intra-operatively—and in real-time or near real-time—measuring leg length and offset during a hip joint replacement procedure. As illustrated in FIG. 4, system 300 may include a processing device (such as processing and display unit 350 (or other computer device for processing data received by system 300)), and one or more wireless communication transceivers 360 for communicating with the magnetic/IMU sensors attached to the patient's anatomy (not shown). The components of system 300 described above are exemplary only, and are not intended to be limiting. Indeed, it is contemplated that additional and/or different components may be included as part of system 300 without departing from the scope of the present disclosure. For example, although wireless communication transceiver 360 is illustrated as being a standalone device, it may be integrated within one or more other components, such as processing and display unit 350. Thus, the configuration and arrangement of components of system 300 illustrated in FIG. 4 are intended to be exemplary only.

Processing and display unit 350 may include or embody any suitable microprocessor-based device configured to process and/or analyze information indicative of relative positions of adjacent bones. According to one embodiment, processing and display unit 350 may be a general purpose computer programmed with software for receiving, processing, and displaying information indicative of the position of the femur relative to the pelvis. According to other embodiments, processing and display unit 350 may be a special-purpose computer, specifically designed to communicate with, and process information for, other components associated with system 300. Individual components of, and processes/methods performed by, processing and display unit 350 will be discussed in more detail below.

Processing and display unit 350 may be communicatively coupled to the sensor module(s) (and any additional orientation sensors (not shown) used in system 300) and may be configured to receive, process, and/or analyze data measured by the modules 340 and 380. According to one embodiment, processing and display unit 350 may be wirelessly coupled to modules 340 and 380 via wireless communication transceiver(s) 360 operating any suitable protocol for supporting wireless (e.g., wireless USB, ZigBee, Bluetooth, Wi-Fi, etc.) In accordance with another embodiment, processing system 350 may be wirelessly coupled to modules 340 and 380, which, in turn, may be configured to collect data from the other constituent sensors and deliver it to processing and display unit 350. In accordance with yet another embodiment, certain components of processing and display unit 350 (e.g. I/O devices 356) may be suitably miniaturized for integration with modules 340 and/or 380.

Wireless communication transceiver(s) 360 may include any device suitable for supporting wireless communication between one or more components of system 300. As explained above, wireless communication transceiver(s) 360 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 360 may embody a standalone communication module, separate from processing and display unit 350. As such, wireless communication transceiver 360 may be electrically coupled to processing and display unit 350 via USB or other data communication link and configured to deliver data received therein to processing and display unit 350 for further processing/analysis. According to other embodiments, wireless communication transceiver 360 may embody an integrated wireless transceiver chipset, such as the Bluetooth, Wi-Fi, NFC, or 802.11x wireless chipset included as part of processing and display unit 350.

As explained, processing and display unit 350 may be any processor-based computing system that is configured to receive placement parameters associated with an orthopedic joint 110, store anatomic registration information, analyze the received placement parameters to extract data indicative of the placement of prosthetic components of orthopedic joint 110 with respect to the patient's anatomy, and output the extracted data in real-time or near real-time. Non-limiting examples of processing and display unit 350 include a desktop or notebook computer, a tablet device, a smartphone, wearable or handheld computers, or any other suitable processor-based computing system.

For example, as illustrated in FIG. 4, processing system 350 may include one or more hardware and/or software components configured to execute software programs, such as software tracking placement parameters associated with a prosthetic component of orthopedic joint 110 and displaying information indicative of the placement of the component. According to one embodiment, processing and display unit 350 may include one or more hardware components such as, for example, a central processing unit (CPU) or microprocessor 351, a random access memory (RAM) module 352, a read-only memory (ROM) module 353, a memory or data storage module 354, a database 355, one or more input/output (I/O) devices 356, and an interface 357. Alternatively and/or additionally, processing and display unit 350 may include one or more software media components such as, for example, a computer-readable medium including computer-executable instructions for performing methods consistent with certain disclosed embodiments. It is contemplated that one or more of the hardware components listed above may be implemented using software. For example, storage 354 may include a software partition associated with one or more other hardware components of processing and display unit 350. Processing and display unit 350 may include additional, fewer, and/or different components than those listed above. It is understood that the components listed above are exemplary only and not intended to be limiting.

CPU 351 may include one or more processors, each configured to execute instructions and process data to perform one or more functions associated with processing and display unit 350. As illustrated in FIG. 4, CPU 351 may be communicatively coupled to RAM 352, ROM 353, storage 354, database 355, I/O devices 356, and interface 357. CPU 351 may be configured to execute sequences of computer program instructions to perform various processes, which will be described in detail below. The computer program instructions may be loaded into RAM 352 for execution by CPU 351.

RAM 352 and ROM 353 may each include one or more devices for storing information associated with an operation of processing and display unit 350 and/or CPU 351. For example, ROM 353 may include a memory device configured to access and store information associated with processing and display unit 350, including information for identifying, initializing, and monitoring the operation of one or more components and subsystems of processing and display unit 350. RAM 352 may include a memory device for storing data associated with one or more operations of CPU 351. For example, ROM 353 may load instructions into RAM 352 for execution by CPU 351.

Storage 354 may include any type of mass storage device configured to store information that CPU 351 may need to perform processes consistent with the disclosed embodiments. For example, storage 354 may include one or more magnetic and/or optical disk devices, such as hard drives, CD-ROMs, DVD-ROMs, or any other type of mass media device. Alternatively or additionally, storage 314 may include flash memory mass media storage or other semiconductor-based storage medium.

Database 355 may include one or more software and/or hardware components that cooperate to store, organize, sort, filter, and/or arrange data used by processing and display unit 350 and/or CPU 351. For example, database 355 may include historical data such as, for example, stored placement data associated with the orthopedic joint. CPU 351 may access the information stored in database 355 to provide a comparison between previous joint component placement and current (i.e., real-time) placement data. CPU 351 may also analyze current and previous placement parameters to identify trends in historical data. These trends may then be recorded and analyzed to allow the surgeon or other medical professional to compare the placement parameters with different prosthesis designs and patient demographics. It is contemplated that database 355 may store additional and/or different information than that listed above.

I/O devices 356 may include one or more components configured to communicate information with a user associated with system 300. For example, I/O devices may include a console with an integrated keyboard and mouse to allow a user to input parameters associated with processing and display unit 350. I/O devices 356 may also include a display including a graphical user interface (GUI) (such as GUI 800 shown in FIG. 8) for outputting information on a display monitor 358a. In certain embodiments, the I/O devices may be suitably miniaturized and integrated with tool 310. I/O devices 356 may also include peripheral devices such as, for example, a printer 358b for printing information associated with processing and display unit 350, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 357 may include one or more components configured to transmit and receive data via a communication network, such as the Internet, a local area network, a workstation peer-to-peer network, a direct link network, a wireless network, or any other suitable communication platform. For example, interface 357 may include one or more modulators, demodulators, multiplexers, demultiplexers, network communication devices, wireless devices, antennas, modems, and any other type of device configured to enable data communication via a communication network. According to one embodiment, interface 357 may be coupled to or include wireless communication devices, such as a module or modules configured to transmit information wirelessly using Wi-Fi or Bluetooth wireless protocols. Alternatively or additionally, interface 357 may be configured for coupling to one or more peripheral communication devices, such as wireless communication transceiver 360.

As explained, system consists of a module 380 comprising at least two 3-axis magnetic sensors that measure the direction and intensity of a magnetic field. These 3-axis magnetic sensors are used to measure the direction and intensity of the magnetic field emanating from the magnetic field generator 375 in module 340. These multiple magnetic sensors are arranged at a known fixed spatial distance and orientation with respect to each other. For example they can be arranged with their X, Y, Z axis aligned and separated at a fixed distance along the X axis. The modules 340 and 380 may be interchangeably attached to either the pelvis or the femur.

For example the magnetic field generator 375 in module 340 could be a permanent magnet or electromagnet (i.e. a wound coil through which current is passed). In the case of an electromagnet, one or more coils may be utilized to create multiple magnetic fields that are at known orientations to each other. As illustrated in FIG. 4, magnetic field generator may be embedded as part of module 340 that is attached to the patient's femur. The corresponding sensors may be included as part of a second module 380 that is affixed to the patient's pelvis.

Module 340 and 380 may also include one or more subcomponents configured to detect and transmit information that either represents the 3-dimensional orientation or can be used to derive the orientation of the module 340 and 380 (and, by extension, any object that is affixed relative to modules 340 and 380, such as a patient's bone). Module 340 and 380 may embody a device capable of determining a 3-dimensional orientation associated with any body to which module 340 and 380 is attached. According to one embodiment, orientation sensor(s) in module 340 and 380 may be an inertial measurement unit including a microprocessor 341, a power supply 342, and one or more of a gyroscope 343, an accelerometer 344, or a magnetometer 345.

According to one embodiment, the inertial measurement units in 340 and 380 may contain a 3-axis gyroscope 343, a 3-axis accelerometer 344, and a 3-axes magnetometer 345. It is contemplated, however, that fewer of these devices with fewer axes can be used without departing from the scope of the present disclosure. For example, according to one embodiment, inertial measurement units may include only a gyroscope and an accelerometer, the gyroscope for calculating the orientation based on the rate of rotation of the device, and the accelerometer for measuring earth's gravity and linear motion, the accelerometer providing corrections to the rate of rotation information (based on errors introduced into the gyroscope because of device movements that are not rotational or errors due to biases and drifts). In other words, the accelerometer may be used to correct the orientation information collecting by the gyroscope. Similarly the magnetometer 345 can be utilized to measure the earth's magnetic field and can be utilized to further correct gyroscope errors. Thus, while all three of gyroscope 343, accelerometer 344, and magnetometer 345 may be used, orientation measurements may be obtained using as few as one of these devices. The use of additional devices increases the resolution and accuracy of the orientation information and, therefore, may be advantageous when orientation accuracy is important.

As illustrated in FIG. 4, microprocessor 341 of modules 340 and 380 may include different processing modules or cores, which may cooperate to perform various processing functions. For example, microprocessor 341 may include, among other things, an interface 341d, a controller 341c, a motion processor 341b, and signal conditioning circuitry 341d. Controller 341c may be configured to control the magnetic field generator 375 which could be based on instructions received from the processor 350 via interface 341d. Controller 341c may also be configured to control and receive conditioned and processed data from one or more of gyroscope 343, accelerometer 344, magnetometer 345, and magnetic sensors 370 and transmit the received data to one or more remote receivers. The data may be pre-conditioned via signal conditioning circuitry 341a, which includes amplifiers and analog-to-digital converters or any such circuits. The signals may be further processed by a motion processor 341b. Motion processor 341b may be programmed with so-called “motion fusion” algorithms to collect and process data from different sensors to generate error corrected orientation information. The orientation information may be a mathematically represented as an orientation or rotation quaternion, euler angles, direction cosine matrix, rotation matrix of any such mathematical construct for representing orientation known in the art. Accordingly, controller 341c may be communicatively coupled (e.g., wirelessly via interface 341d as shown in FIG. 4, or using a wireline protocol) to, for example, processing and display unit 350 and may be configured to transmit the orientation data received from one or more of gyroscope 343, accelerometer 344, and magnetometer 345 to processing and display unit 350, for further analysis.

Interface 341d may include one or more components configured to transmit and receive data via a communication network, such as the Internet, a local area network, a workstation peer-to-peer network, a direct link network, a wireless network, or any other suitable communication platform. For example, interface 341d may include one or more modulators, demodulators, multiplexers, demultiplexers, network communication devices, wireless devices, antennas, modems, and any other type of device configured to enable data communication via a communication network. According to one embodiment, interface 341d may be coupled to or include wireless communication devices, such as a module or modules configured to transmit information wirelessly using Wi-Fi or Bluetooth wireless protocols. As illustrated in FIG. 4, modules 340 and 380 may be powered by power supply 342, such as a battery, fuel cell, MEMs micro-generator, or any other suitable compact power supply.

Importantly, although microprocessor 341 of module 340 and 380 is illustrated as containing a number of discrete modules, it is contemplated that such a configuration should not be construed as limiting. Indeed, microprocessor 341 may include additional, fewer, and/or different modules than those described above with respect to FIG. 4, without departing from the scope of the present disclosure. Furthermore, in other instances of the present disclosure that describe a microprocessor are contemplated as being capable of performing many of the same functions as microprocessor 341 of modules 340 and 380 (e.g., signal conditioning, wireless communications, etc.) even though such processes are not explicitly described with respect to microprocessor 341. Those skilled in the art will recognize that many microprocessors include additional functionality (e.g., digital signal processing functions, data encryption functions, etc.) that are not explicitly described here. Such lack of explicit disclosure should not be construed as limiting. To the contrary, it will be readily apparent to those skilled in the art that such functionality is inherent to processing functions of many modern microprocessors, including the ones described herein.

Microprocessor 341 may be configured to receive data from one or more of gyroscope 343, accelerometer 344, magnetometer 345, and magnetic sensors 370, and transmit the received data to one or more remote receivers. Accordingly, microprocessor 341 may be communicatively coupled (e.g., wirelessly (as shown in FIG. 4, or using a wireline protocol) to, for example, processing and display unit 350 and configured to transmit the orientation and position data received from one or more of gyroscope 343, accelerometer 344, magnetometer 345, and magnetic sensors 370 to processing and display unit 350, for further analysis. As illustrated in FIG. 4, microprocessor 341 may be powered by power supply 342, such as a battery, fuel cell, MEMs micro-generator, or any other suitable compact power supply.

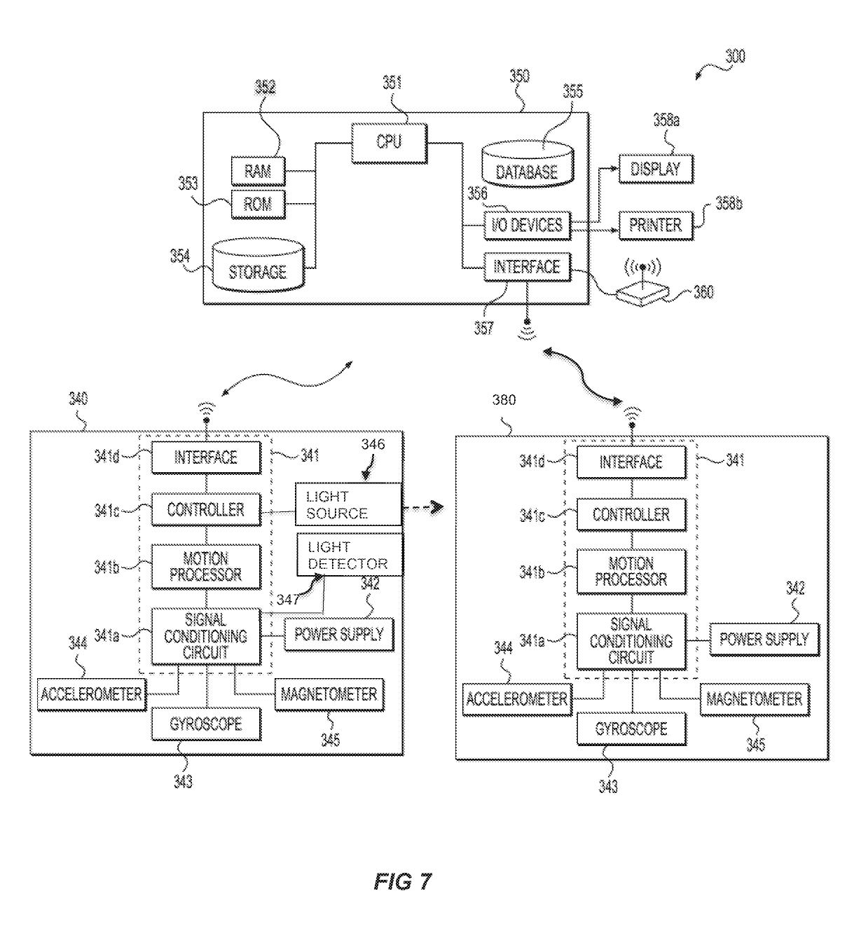

An alternate embodiment, illustrated in FIG. 7, utilizes an optical distance measurement system in lieu of or in addition to the magnetic method. Module 340 comprises a light source 346 that is directed towards module 380 and light detector 347 that measures the light reflected from module 380. Alternatively, the light detector 347 can be housed in module 380 and measures the light emitted from source 346 in module 340. The light source can be any suitable light source such as a laser or light emitting diode that allows for measurement of distance using one of several optical distance measuring methods known in the art. Examples of such methods are intensity, interferometry, triangulation, Doppler, and time-of-flight (TOF). The light detector 347 can be any suitable light detector such as a photo resistor, photo diode, photo transistor, photo voltaic cell, etc., with associated signal processing circuitry. Alternatively, the light detector 347 can be a miniaturized camera.

Anatomic Registration

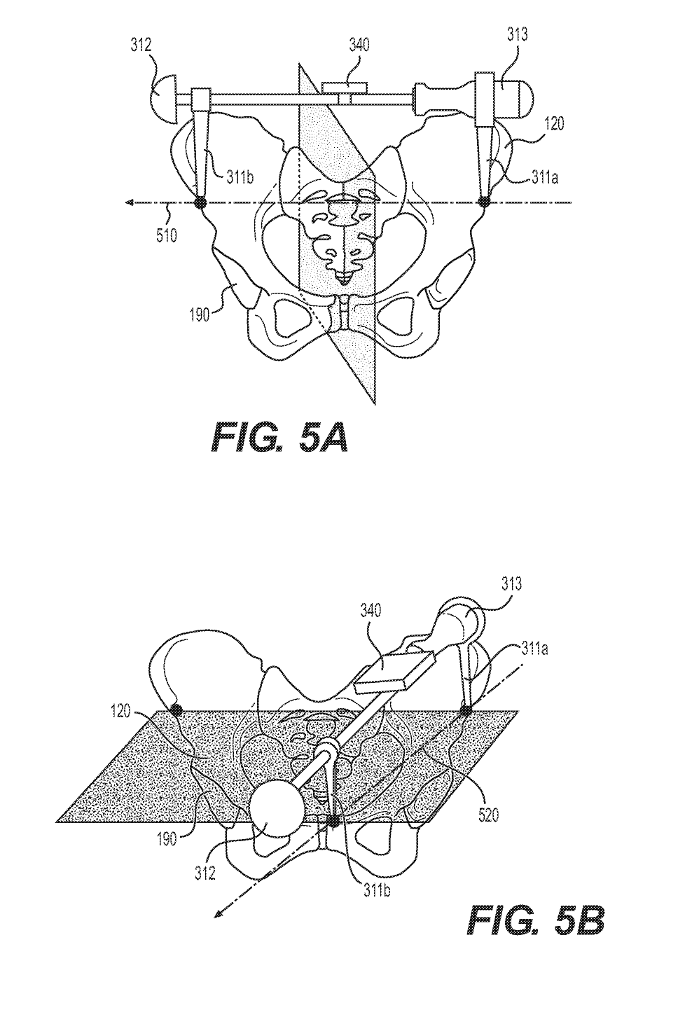

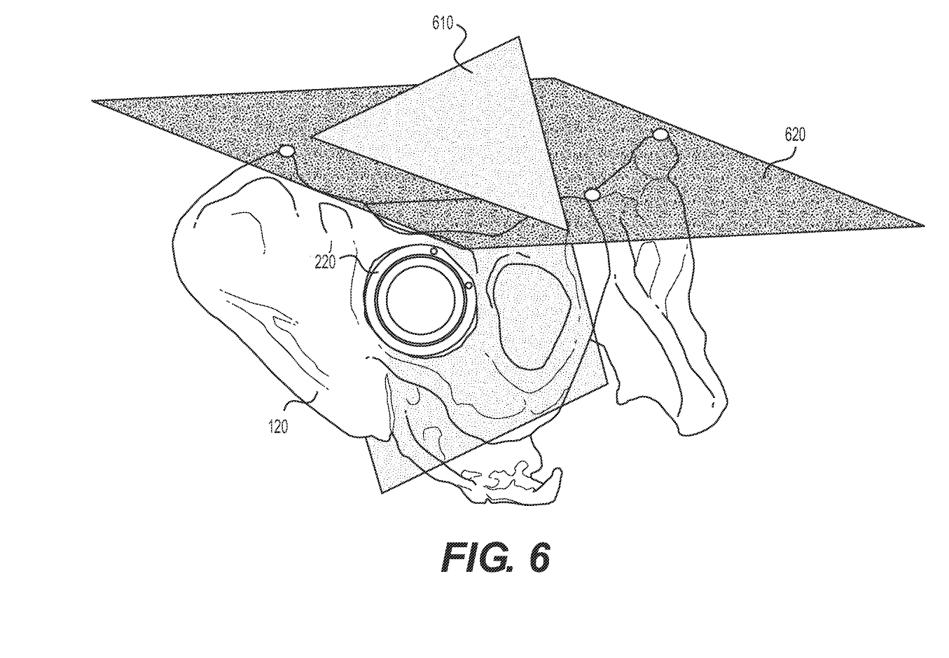

As explained, in order for system 300 to accurately estimate the leg length and offset, it must register the virtual coordinate system of the patient's pelvis. This allows the system to convert changes in the relative positions of the adjacent bones into the appropriate anatomical components such as the inferior-superior (Leg length) and medial-lateral (Offset) components. Modules 340 and 380 have their own X, Y, Z coordinate system and the process of registration establishes the relationship between the modules' coordinate system and the patient's anatomy. The term “virtual,” as is used herein refers to a plane, vector, or coordinate system that exists as a mathematical or algorithmic representation within a computer software program. In other words, “virtual coordinate system” refers to an algorithmic mapping of points within an environment to a particular object, such as a bone or other portion of the patient's anatomy. To estimate the leg length and offset, system 300 is configured to measure an orientation of the longitudinal axis of registration tool 313 using an attached orientation sensor in different positions relative to certain anatomical landmarks associated with the patient's pelvic anatomy. Using geometrical relationships associated with the anatomical landmarks, the information indicative of the orientation of registration tool 313 can be used to derive a virtual coordinate space that is representative of the pelvis, and associate modules 340 and/or 380 with this virtual coordinate space. FIGS. 5A, 5B, and 6, illustrate an exemplary process for establishing a virtual coordinate space for the pelvis, registering module 340 and/or 380 to the virtual coordinate space in accordance with the disclosed embodiments. Only module 340 is shown for illustration purposes.

A common pelvic reference plane is the anterior pelvic plane (illustrated as plane 620 of FIG. 6 and the plane of FIG. 5B) which is defined by the locations of the left and right anterior superior iliac spines (ASIS) and pubic symphsis. The saggital plane (illustrated as plane 610 in FIG. 6 and the plane of FIG. 5A) is perpendicular to the anterior pelvic plane. According to one embodiment, once module 340 and/or 380 have been registered/calibrated to these anatomical planes of pelvis 120, modules 340 and 380 can be used to estimate the leg length and offset in real-time. Importantly, although the processes described in accordance with certain exemplary embodiments used the saggital and anterior pelvic planes to create a virtual coordinate system for the patient's anatomy, it is contemplated that any number of anatomical calibration techniques and landmarks can be used to determine the virtual pelvic coordinate space. For example, it is contemplated that certain bony landmarks of the pelvis can be used to determine the orientation of the transverse pelvic plane and this plan can be used as a basis for registering modules 340 and/or 380. Consequently, any of a number of different combinations of reference points/planes that can be used to define a virtual coordinate system of pelvis 120 and subsequently register modules 340 and/or 380 to the virtual coordinate system without departing from the scope of the present disclosure.

One exemplary process for registering module 340 and/or 380 to a virtual coordinate system associated with pelvis 120 commences by removably attaching module 340 and/or 380 to the registration tool 313 and receiving, at processing system 350 from orientation sensors in module 340 and/or 380, information indicative of a first orientation between estimated positions of left and right anterior superior iliac spines (ASIS). FIG. 5A illustrates an exemplary embodiment for using tool 313 to measure the information indicative of the first orientation.

As illustrated in FIG. 5, pointers 311a, 311b of tool 313 are placed at portions of the patient's anatomy that correspond to the left and right ASIS of pelvis 120. In this position, the orientation sensor attached to the registration tool 313 measures the orientation associated with tool 313, which correspond to the orientation of a virtual axis that passes through the 2 ASIS's. During a surgical procedure, pointers 311a, 311b are brought in contact with a patient's anatomy corresponding to estimated positions of the anatomical landmarks of pelvis 120 (in an exemplary embodiment, the left and right anterior superior iliac spines (ASIS)). When the user is satisfied with the position of pointers 311a, 311b, the orientation associated with registration tool 313 is measured by the attached module 340 or 380 and transmitted to processing system 350 for storage. One or more points or vectors maybe be recorded and averaged to improve accuracy. The recorded orientation is parallel to the frontal horizontal axis of the body (axis that passes from side to side). Using mathematical formulas based on geometry the processing unit is then able to calculate the orientation of a plane that is perpendicular to this recorded orientation. This perpendicular plane is parallel to saggital plane (610 of FIG. 6) and its orientation is indicative of the orientation of saggital plane 610.

The registration process continues by receiving, at processing system 350 from the attached module 340 or 380 of the registration tool, information indicative of the orientation of a second virtual axis established between at least one of the estimated positions of the left and right anterior superior iliac spines and one of left or right pubic symphsis. As illustrated in FIG. 5B, for example, pointers 311a, 311b are placed on one of the left or right ASIS and one of the left or right pubic symphsis. In this position, the module attached to the registration tool measures the orientation of a second virtual axis that passes through those points. The orientation of this axis relative to the axis recorded earlier is calculated. Since the three anatomic landmarks used in the registration process lie on the anterior pelvic plane, the two virtual axes recorded in the earlier steps are parallel to the anterior pelvic plane (and non-parallel to one another). The orientation of the anterior pelvic plan can be therefore be calculated by the processing unit using mathematical formulas based on geometry. In both FIGS. 5A and 5B, a dome shaped cup 312 is depicted. The dome shaped cup 312 is optional and not necessary for the particular embodiments described above.

According to the exemplary embodiment, once the first virtual plane (indicative of a plane parallel with saggital plane 610) and the second virtual plant (indicative of a plane parallel with the anterior pelvic plane 620) have been determined, processing system 350 registers/calibrates module 340 and/or 380 to the patient's virtual pelvic coordinate space and stores that information. According to one embodiment, processing system 350 is configured to mathematically transform the raw orientation measurements from orientation sensor of module 340 and/or 380 to an orientation angle relative to either or both of the first and second virtual planes. After registration of the pelvic coordinate frame, modules 340 and/or 380 are removed from the registration tool and rigidly attached to the patient's anatomy. Their orientation and position when attached to the anatomy is also registered as a reference by processing and display unit 350. It is not necessary to register both module 340 and 380 to the anatomy since the modules may be calibrated to establish their mutual relationship and therefore only the relationship of one of the modules with respect to the patient's anatomy needs to be established via the registration process above. Similarly, it is also possible to register the anatomy using a third module attached to the registration tool that does not need to be removed and attached to the patient's bone after registration.

Although certain exemplary embodiments do not rely on any pre-operative or intra-operative imaging data, certain embodiments consistent with the present disclosure may be used in conjunction with such information. For example, if the surgeon is unable to reliably find and point to the bony landmarks (e.g. in the case of an obese patient) imaging data (such as x-ray or CT scan data) can be used to aid in completing the above-outlined registration process. For example, although the ASIS landmarks are easily and reliably found even in obese patients, palpating and pointing to the pubic symphsis can be challenging. In such situations, instead using the pubic symphsis to determine the second plane, pelvic tilt data (i.e., the tilt associated with the anterior pelvic plane with respect to the frontal (coronal) plane of the body) may be determined using imaging techniques (such as a lateral X-ray) either pre-operatively or intra-operatively. Any other imaging modality such as MRI and CT-scan may also be used to get this information. This pelvic tilt information may be input to the processing unit and this along with the first registration of the ASIS is sufficient to determine the orientation of the anterior pelvic plane, without having to palpate and/or point to the pubic symphsis.

In one embodiment of the current invention, a reference sensor is embedded into or attached to a patient specific instrument. The orientation of the reference sensor with respect to the patient specific instrument is known either from design or measured during the manufacturing process of the patient-specific instrument. Alternatively, the reference sensor can be attached to the patient-specific instrument intra-operatively at a known orientation using mating features on the patient-specific instrument or alignment marks. Also, as previously mentioned, the patient specific instrument is designed for fixation to the patient's anatomy at a pre-determined anatomic orientation. With the above two relationships known, the relative orientation of the reference sensor with respect to the patient's anatomy can then be derived. In effect the reference sensor is pre-operatively registered to the patient's anatomy using the patient-specific instrument as a vehicle (regardless of when the reference sensor is actually attached). Such “pre-registration” eliminates the need for manual registration of the anatomy and results in a system this is truly “point and shoot.”

Processes and methods consistent with the disclosed embodiments have been described in accordance with specific joint replacement procedures, namely a hip joint replacement procedure. This skilled in the art will recognize, however, that these descriptions were exemplary only, and that the presently disclosed prosthetic placement tracking system—using a technique that involves either manual tool registration/calibration with a patient's anatomy or a patient-specific registration technique—can be used in most any situation in which precise placement of a prosthetic component is important.

It will be apparent to those skilled in the art that various modifications and variations can be made to the disclosed systems and methods for measuring orthopedic placement parameters associated with a reconstructed joint in orthopedic arthroplastic procedures. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

1. A method for estimating leg length and offset, comprising:
registering an anatomic coordinate frame associated with a patient's pelvis;
receiving, from at least two magnetic sensors, information indicative of a first position of a femur relative to the patient's pelvis;
receiving, from the at least two magnetic sensors, information indicative of a change in a position of the femur relative to the patient's pelvis; and
determining at least one of a leg length and an offset change based on the first position and the change in position.

2. The method of claim 1, wherein registering the anatomic coordinate frame associated with the patient's pelvis includes calculating an orientation of at least one of the anterior pelvic plane or a plane parallel to the anterior pelvic plane.

3. The method of claim 1, wherein registering the anatomic coordinate frame associated with the patient's pelvis includes calculating an orientation of at least one of the saggital plane or a plane parallel to the saggital plane.

4. The method of claim 1, further comprising receiving, from at least two orientation sensors, information indicative of the first position and/or information indicative of a change in the position.

5. A system for estimating leg length and offset associated with a patient's joint, comprising:
a magnetic field generator coupled to a patient's femur or pelvis at a first location and configured to generate a magnetic field;
a plurality of magnetic sensors coupled to the adjacent bone and configured to measure the relative strength and direction of the magnetic field at this second location;
orientation sensors coupled to both the first and second locations and configured to detect information indicative of an orientation of the locations relative to the patient's anatomy as well relative orientation between the first and second locations; and
a processor, communicatively coupled to the orientation sensors and magnetic sensors and configured to:
register an anatomic coordinate frame associated with a patient's pelvis;
measure a first position of a femur relative to the patient's pelvis;
receive information indicative of a change in a position of the femur relative to the patient's pelvis; and
determine at least one of a leg length and a offset based on the first position and the change in position.

6. The system of claim 5, wherein the orientation sensors are inertial measurement units that include at least one of a gyroscope, an accelerometer, or a magnetometer.

7. The system of claim 5, wherein the orientation sensor includes a gyroscope and an accelerometer.

8. The system of claim 5, wherein each of the plurality of magnetic sensors includes a 3-axis magnetic sensor.

9. The system of claim 5, wherein the magnetic field generator is an electromagnet.

10. The system of claim 5, wherein the magnetic field generator is a permanent magnet.

11. A method for estimating leg length and offset, comprising:
registering an anatomic coordinate frame associated with a patient's pelvis;
receiving, from optical sensors, information indicative of a first position of a femur relative to the patient's pelvis;
receiving, from optical sensors, information indicative of a change in a position of the femur relative to the patient's pelvis; and
determining at least one of a leg length and a offset change based on the first position and the change in position.

12. The method of claim 11, further comprising receiving from orientation sensors information indicative of the first position and the change in position.

13. The method of claim 11, wherein registering the anatomic coordinate frame associated with the patient's pelvis includes calculating an orientation of at least one of the anterior pelvic plane or a plane parallel to the anterior pelvic plane.

14. The method of claim 11, wherein registering the anatomic coordinate frame associated with the patient's pelvis includes calculating an orientation of at least one of the saggital plane or a plane parallel to the saggital plane.

15. A system for estimating leg length and offset associated with a patient's joint, comprising:
a light source coupled to a patient's femur or pelvis at a first location and configured to point in the general direction of the adjacent bone;
a light detector configured to measure light reflected from the adjacent bone or a light detector coupled to the adjacent bone and configured to measure the light received at this second location;
orientation sensors coupled to both the first and second locations and configured to detect information indicative of an orientation of the locations relative to the patient's anatomy as well relative orientation between the first and second locations; and
a processor, communicatively coupled to the orientation sensors and light detector and configured to:
register an anatomic coordinate frame associated with a patient's pelvis;
measure a first position of a femur relative to the patient's pelvis;
receive information indicative of a change in a position of the femur relative to the patient's pelvis; and
determine at least one of a leg length and a offset based on the first position and the change in position.

16. The system of claim 15, wherein each orientation sensor is an inertial measurement unit that includes at least one of a gyroscope, an accelerometer, or a magnetometer.

17. The system of claim 15, wherein the orientation sensor includes a gyroscope and an accelerometer.

18. The system of claim 15, wherein each of the plurality of magnetic sensors includes a 3-axis magnetic sensor.

19. The system of claim 15, wherein the light source is a light emitting diode.

20. The system of claim 15, wherein the light source is a laser.

21. A robotic surgical system, comprising:
a processor and a memory communicatively connected to the processor, wherein the processor is configured to:
register an anatomic coordinate frame associated with a patient's pelvis;
receive, from at least two magnetic sensors, information indicative of a first position of a femur relative to the patient's pelvis;
receive, from the at least two magnetic sensors, information indicative of a change in a position of the femur relative to the patient's pelvis; and
determine at least one of a leg length and an offset change based on the first position and the change in position, wherein the robotic surgical system is configured to treat a degenerative disease or deformity of an orthopedic or spinal structure.
